(19) United States
(12) Patent Application Publication
(10) Pub. No.: US 2026/0225098 A1
(43) Pub. Date: Aug. 6, 2026

(54) PARTICLE SEPARATION APPARATUS AND PARTICLE SEPARATION METHOD

(71) Applicant: CANON KABUSHIKI KAISHA, Tokyo (JP)

(72) Inventors: Tsutomu MIKI, Komae (JP); Yuhi HIGUCHI, Tokyo (JP)

(73) Assignee: CANON KABUSHIKI KAISHA, Tokyo (JP)

(21) Appl. No.: 19/574,984

(22) Filed: Mar. 23, 2026

Related U.S. Application Data
(63) Continuation of application No. PCT/JP2024/035661, filed on Oct. 4, 2024.

(30) Foreign Application Priority Data
Oct. 6, 2023 (JP) 2023-174692

(51) Int. Cl. B01L 3/00; B01D 21/00

(52) U.S. Cl. CPC B01L 3/502753; B01D 21/0057; B01L 2200/0652; B01L 2300/0681; B01L 2300/0816; B01L 2300/0864; B01L 2300/0883

(57) ABSTRACT

According to one embodiment, a particle separation apparatus includes a container, a filter, and a microchannel. The container is configured to store a suspension liquid containing target particles and non-target particles smaller than the target particles. The filter is configured to catch the target particles from the suspension liquid and allow the non-target particles to pass through. The microchannel is configured to separate the target particles from the suspension liquid. The suspension liquid stored in the container is sent to the filter. The suspension liquid containing the target particles caught by the filter is sent to the microchannel.

### CROSS-REFERENCE TO RELATED APPLICATION(S

This application is a Continuation Application of PCT Application No. PCT/JP2024/035661, filed October 4, 2024 and based upon and claiming the benefit of priority from prior Japanese Patent Application No. 2023-174692, filed October 6, 2023, the entire contents of all of which are incorporated herein by reference.

### FIELD

Embodiments described herein relate generally to a particle separation apparatus and a particle separation method.

### BACKGROUND

Manufacturing of induced pluripotent stem (iPS) cells requires white blood cells. To separate white blood cells (an example of target particles) from whole blood, red blood cells (an example of non-target particles) which make up a large part of whole blood need to be removed.

For example, there is a method in which the whole blood is diluted with a saline solution (PBS), and subsequently white blood cells are separated from whole blood after a dilution process. However, because the dilution process increases the volume of the whole blood, a process time required for separating white blood cells also increases.

### BRIEF DESCRIPTION OF THE DRAWINGS

FIG. 1 is a schematic diagram showing a configuration example of a particle separation apparatus according to an embodiment.

FIG. 2 is a schematic view showing an external appearance of a spiral flow channel according to the present embodiment.

FIG. 3 is a flowchart showing an operation example of the particle separation apparatus according to the present embodiment.

FIG. 4 is a schematic view showing blood suction according to the present embodiment.

FIG. 5 is a schematic diagram showing blood filtering according to the present embodiment.

FIG. 6 is a schematic view showing a primary PBS suction according to the present embodiment.

FIG. 7 is a schematic view showing PBS rinsing according to the present embodiment.

FIG. 8 is a schematic diagram showing a secondary PBS suction according to the present embodiment.

FIG. 9 is a schematic view showing cell collection according to the present embodiment.

FIG. 10 is a schematic diagram showing a primary classification according to the present embodiment.

FIG. 11 is a schematic diagram showing a secondary classification according to the present embodiment.

### DETAILED DESCRIPTION

In general, according to one embodiment, a particle separation apparatus includes a container, a filter, and a microchannel. The container is configured to store a suspension liquid containing target particles and non-target particles smaller than the target particles. The filter is configured to catch the target particles from the suspension liquid and allow the non-target particles to pass through. The microchannel is configured to separate the target particles from the suspension liquid. The suspension liquid stored in the container is sent to the filter. The suspension liquid containing the target particles caught by the filter is sent to the microchannel.

Hereinafter, an embodiment will be described with reference to the drawings. In the present embodiment, parts assigned the same reference signs are assumed to perform the same operations, and redundant descriptions will be omitted as appropriate.

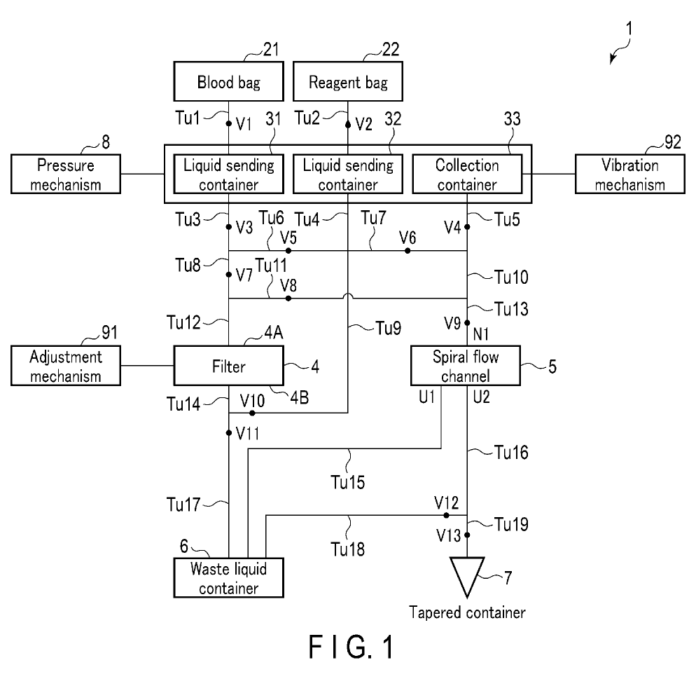

FIG. 1 is a schematic diagram illustrating a configuration example of a particle separation apparatus 1 according to the present embodiment. FIG. 1 shows the particle separation apparatus 1 as seen in a horizontal direction. The particle separation apparatus 1 is an apparatus for separating target particles. The particle separation apparatus 1 includes a blood bag 21, a reagent bag 22, liquid sending containers 31 and 32, a collection container 33, a filter 4, a spiral flow channel 5, a waste liquid container 6, a tapered container 7, a pressure mechanism 8, an adjustment mechanism 91, a vibration mechanism 92, tubes Tu1 to Tu19, and valves V1 to V13.

The blood bag 21 is a bag to store blood (whole blood). The blood bag 21 is connected to the liquid sending container 31 by the tube Tu1. The blood bag 21 is an example of a reservoir (blood reservoir).

The reagent bag 22 is a bag to store a reagent (for example, PBS). The reagent bag 22 is connected to the liquid sending container 32 by the tube Tu2. The reagent bag 22 is an example of a reservoir (reagent reservoir).

The liquid sending container 31 (first liquid sending container) is a container to temporarily store the liquid to be sent. The liquid sending container 31 temporarily stores blood. The liquid sending container 31 is connected to the filter 4 (particularly, a trapping surface 4A) by the tubes Tu3, Tu8, and Tu12. The liquid sending container 31 is connected to the spiral flow channel 5 by the tubes Tu3, Tu6, Tu7, Tu10, and Tu13. The liquid sending container 31 is an example of a reservoir (blood reservoir).

The liquid sending container 32 (second liquid sending container) is a container to temporarily store a liquid to be sent. The liquid sending container 32 temporarily stores a reagent. The liquid sending container 32 is connected to the filter 4 (particularly, the trapping surface 4A) by the tubes Tu4, Tu6, Tu8, and Tu12. The liquid sending container 32 is connected to the filter 4 (particularly, an opposite surface 4B) by the tubes Tu4, Tu9, and Tu14. The liquid sending container 32 is connected to the spiral flow channel 5 by the tubes Tu4, Tu7, Tu10, and Tu13. The liquid sending container 32 is an example of a reservoir (reagent reservoir).

The collection container 33 is a container to temporarily store the collected liquid. The collection container 33 stores a suspension liquid containing white blood cells caught by the filter 4 (particularly, the trapping surface 4A). The collection container 33 is connected to the filter 4 (particularly, the trapping surface 4A) by the tubes Tu5, Tu10, Tu11, and Tu12. The collection container 33 is connected to the spiral flow channel 5 (particularly, an inlet N1) by the tubes Tu5, Tu10, and Tu13. The collection container 33 is an example of a reservoir (suspension liquid reservoir).

The filter 4 is a filter to catch target particles from the suspension liquid and to allow non-target particles smaller than the target particles to pass through. The filter 4 catches white blood cells from blood containing white blood cells, red blood cells, platelets, and the like, and allows the red blood cells, platelets, and the like smaller than the white blood cells to pass through. The filter 4 has a large number of pores. Preferably, the pore size is smaller than the particle size of the target particles and larger than the particle size of the non-target particles. The filter 4 may be formed of a nonwoven fabric. As the filter 4, for example, a white blood cell removal filter such as Acrodisc WBC syringe filter (trade name: AP-4951) manufactured by Pall Corporation or Sepacell RZ (trade name: RZ-1000N, RZ-2000N) manufactured by Asahi Kasei Medical Co., Ltd. can be appropriately used. The filter 4 is connected to the adjustment mechanism 91. The filter 4 has the trapping surface 4A and the opposite surface 4B. The filter 4 is an example of a trapping part.

The target particle may have a particle size of 7 μm or more and 30 μm or less. The non-target particle may have a particle size of less than 7 μm. The target particle may be a white blood cell. The non-target particle may be a red blood cell, a platelet, etc. In particular, in a case where the target particles and the non-target particles have the above-described particle sizes, the size of the pores of the filter 4 may be 6 μm or more and 7 μm or less.

The trapping surface 4A of the filter 4 is a surface to trap the target particles. Liquids (e.g. blood, a reagent, a suspension liquid) are sent to the trapping surface 4A. The opposite surface 4B of the filter 4 is a surface located on the opposite side of the trapping surface 4A. The liquid that has passed through the trapping surface 4A is sent from the opposite surface 4B. The opposite side 4B is connected to the waste liquid container 6 by the tubes Tu14 and Tu17.

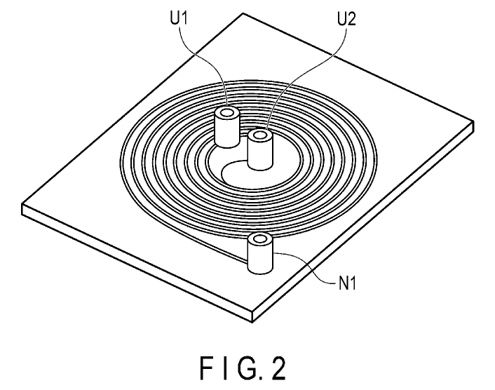

The spiral flow channel 5 is a spiral microchannel (see FIG. 2). The spiral flow channel 5 separates the target particles from the suspension liquid. The spiral flow channel 5 separates white blood cells from blood containing white blood cells, red blood cells, platelets, and the like. The spiral flow channel 5 has one inlet N1, and two outlets U1 and U2. The outlet U1 corresponds to an outer outlet of the microchannel. Particles having a relatively small particle size are sent out from the outlet U1. The outlet U2 corresponds to an inner outlet of the microchannel. Particles having a relatively large particle size are sent out from the outlet U2. The spiral flow channel 5 is an example of a separation part.

A liquid (e.g., blood, a reagent, a suspension liquid) is sent to the inlet N1 of the spiral flow channel 5. From the outlet U1 of the spiral flow channel 5, a suspension liquid mainly containing non-target particles (e.g., red blood cells, platelets) separated by the spiral flow channel 5 is sent out. The outlet U1 is connected to the waste liquid container 6 by the tube Tu15. From the outlet U2 of the spiral flow channel 5, a suspension liquid mainly containing target particles (for example, white blood cells) separated by the spiral flow channel 5 is sent out. The outlet U2 is connected to the waste liquid container 6 by the tubes Tu16 and Tu18. The outlet U2 is connected to the tapered container 7 by the tubes Tu16 and Tu19.

The waste liquid container 6 is a container to store a used liquid (waste liquid). The waste liquid container 6 stores the suspension liquid containing the non-target particles that have passed through the filter 4. The waste liquid container 6 stores the suspension liquid containing the non-target particles separated by the spiral flow channel 5. The waste liquid container 6 is an example of a reservoir (waste liquid reservoir).

According to the present embodiment, one waste liquid container 6 stores the suspension liquid from the filter 4 and stores the suspension liquid from the spiral flow channel 5. Alternatively, one waste liquid container 6 may store the suspension liquid from the filter 4 and another waste liquid container 6 may store the suspension liquid from the spiral flow channel 5. The particle separation apparatus 1 according to the present embodiment uses one waste liquid container 6 instead of two waste liquid containers 6, and thus the entire configuration of the particle separation apparatus 1 can be simplified.

The tapered container 7 is a container having a tapered shape. The tapered container 7 has a shape in which the diameter gradually decreases from the top part to the bottom part. The tapered container 7 stores the suspension liquid mainly containing the target particles separated by the spiral flow channel 5. The tapered container 7 is an example of a reservoir (a suspension liquid reservoir, a separated liquid reservoir).

The pressure mechanism 8 is a mechanism that pressurizes or depressurizes the containers (the liquid sending containers 31 and 32, and the collection container 33). The pressure mechanism 8 pressurizes the container by sending air to an inside of the container. By pressurizing the container, the liquid stored in the container is sent toward a point where the pressure is lower. The pressure mechanism 8 reduces the pressure of the container by suctioning air from the inside of the container. The depressurization of the container causes the liquid to be suctioned into the container from a point where the pressure is higher. That is, the pressure mechanism 8 realizes the sending of the liquid stored in the container and realizes the suctioning of the liquid into the container. The number of the pressure mechanism 8 is not limited to one, and a plurality of pressure mechanisms may be used depending on the application. For example, a pressure mechanism for sending the suspension liquid stored in the reservoir to the filter 4 and a pressure mechanism for sending the suspension liquid containing the target particles caught by the filter 4 to the microchannel may be independently provided. The pressure mechanism 8 is an example of a pressure part, a sending part, or a suction part.

The adjustment mechanism 91 is a mechanism that adjusts a posture (angle) in which the filter 4 is installed. The adjustment mechanism 91 may adjust the posture of the filter 4 to be horizontal. The adjustment mechanism 91 may include a stage to hold the filter 4 and a drive mechanism to adjust a posture of the stage. In this case, the adjustment mechanism 91 adjusts the posture of the filter 4 held on the stage by controlling the drive mechanism to adjust the posture of the stage. The adjustment mechanism 91 is an example of an adjustment part.

According to the present embodiment, the pressure mechanism 8 sends the suspension liquid vertically downward with respect to the filter 4 (particularly, the trapping surface 4A) that has been adjusted to be horizontal by the adjustment mechanism 91. At this time, the suspension liquid that is sent uniformly passes through the trapping surface 4A of the filter 4. Therefore, the filter 4 can efficiently catch the target particles from the suspension liquid that has been sent and can efficiently remove the non-target particles.

The vibration mechanism 92 is a mechanism to vibrate the collection container 33. The vibration mechanism 92 may vibrate the suspension liquid stored in the collection container 33. The vibration mechanism 92 may include a base on which the collection container 33 is placed and a vibration device (vibrator) to vibrate the base. In this case, the vibration mechanism 92 controls the vibration device to vibrate the base, and thus vibrates the collection container 33 placed on the base. The vibration mechanism 92 is an example of a vibration part.

According to the present embodiment, the pressure mechanism 8 sends the suspension liquid vibrated by the vibration mechanism 92 to the spiral flow channel 5. The vibration mechanism 92 vibrates the suspension liquid to release or prevent an aggregation of the particles in the suspension liquid. Thus, the spiral flow channel 5 can effectively separate the target particles and the non-target particles from the vibrated suspension liquid.

The tubes Tu1 to Tu19 are tubes through which a liquid flows. The tubes Tu1 to Tu19 are formed of silicone rubber, polyethylene, metals, or the like. One tube and another tube may be connected to each other by a branch tube (e.g., a Y-tube, a T-tube, a cross-tube). The tubes Tu1 to Tu19 are an example of a connecting part.

The valves V1 to V13 are valves that form a flow path of a liquid that flows through the tubes Tu1 to Tu19. The valves V1 and V13 are valves (bi-directional valves) that allow a liquid to flow in both directions. Each valve is set to an “open state” or a “closed state” manually or automatically. The valves V1 to V13 are an example of a flow channel forming part.

The valves V1 to V13 are arranged in the middle of the tubes Tu1 to Tu19. Hereinafter, each valve and a tube in which each valve is arranged are denoted as (valve name, tube name). According to this notation, the following combinations are obtained: (V1, Tu1), (V2, Tu2), (V3, Tu3), (V4, Tu5), (V5, Tu6), (V6, Tu7), (V7, Tu8), (V8, Tu11), (V9, Tu13), (V10, Tu9), (V11, Tu17), (V12, Tu18), and (V13, Tu19).

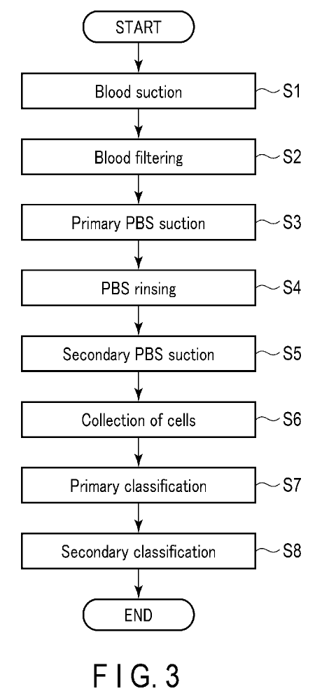

FIG. 3 is a flowchart illustrating an operation example of the particle separation apparatus 1 according to the present embodiment. The particle separation apparatus 1 causes a liquid (for example, blood, a reagent, a suspension liquid) to flow through the tubes Tu1 to Tu19 by controlling the pressure mechanism 8 and the valves V1 to V13.

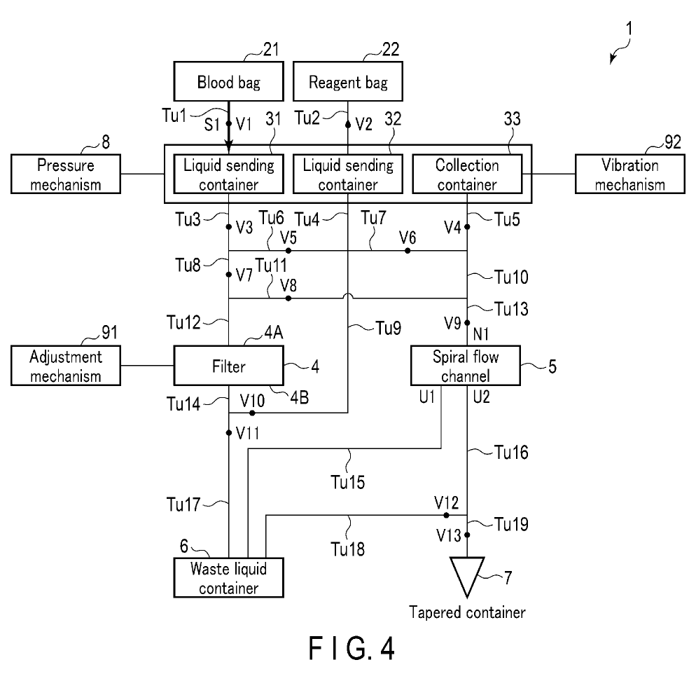

(Step S1) First, the particle separation apparatus 1 performs blood suction (see FIG. 4). More specifically, the particle separation apparatus 1 sets the valve V1 to an “open state” and sets the valves V2 to V13 to a “closed state”. The particle separation apparatus 1 controls the pressure mechanism 8 to depressurize the liquid sending container 31. Thus, the blood stored in the blood bag 21 is sent to the liquid sending container 31 through the tube Tu1.

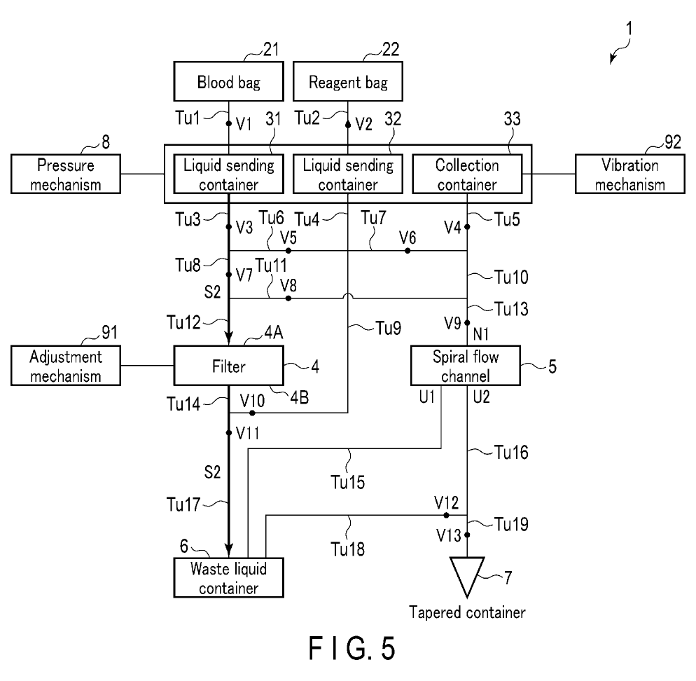

(Step S2) Next, the particle separation apparatus 1 performs blood filtering (see FIG. 5). More specifically, the particle separation apparatus 1 sets the valves V3, V7, and V11 to the “open state”, and sets the valves V1 and V2, V4 to V6, V8 to V10, and V12 to V13 to the “closed state”. The particle separation apparatus 1 controls the pressure mechanism 8 to pressurize the liquid sending container 31. As a result, the blood stored in the liquid sending container 31 is sent to the trapping surface 4A of the filter 4 through the tubes Tu3, Tu8, and Tu12. The filter 4 catches the white blood cells in the blood by the trapping surface 4A and allows the red blood cells, the platelets, and the like to pass through. The blood that has passed through the trapping surface 4A is sent out from the opposite surface 4B of the filter 4, and is sent to the waste liquid container 6 through the tubes Tu14 and Tu17.

The particle separation apparatus 1 may send the blood to the filter 4 at a first flow rate for a predetermined time from the start of sending the blood, and subsequently send the blood to the filter 4 at a second flow rate lower than the first flow rate. For example, the particle separation apparatus 1 sends the blood to the filter 4 at 10 mL/min for 10 seconds from the start of sending the blood, and subsequently sends the blood to the filter 4 at 5 mL/min. This allows the filter 4 to effectively catch white blood cells. That is, the particle separation apparatus 1 can suppress loss of necessary cells.

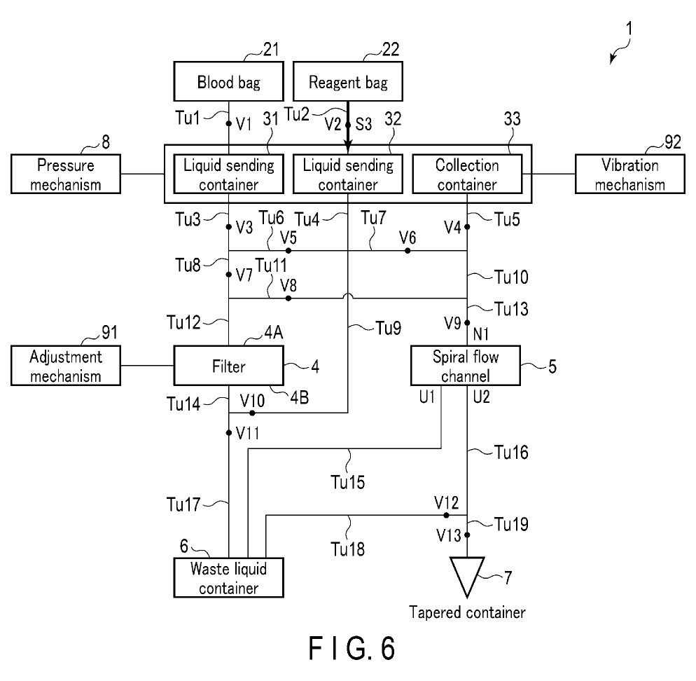

(Step S3) Subsequently, the particle separation apparatus 1 performs a primary PBS suction (see FIG. 6). In particular, the particle separation apparatus 1 sets the valve V2 to the “open state” and the valves V1 and V3 to V13 to the “closed state”. The particle separation apparatus 1 controls the pressure mechanism 8 to depressurize the liquid sending container 32. As a result, the PBS stored in the reagent bag 22 is sent to the liquid sending container 32 through the tube Tu2.

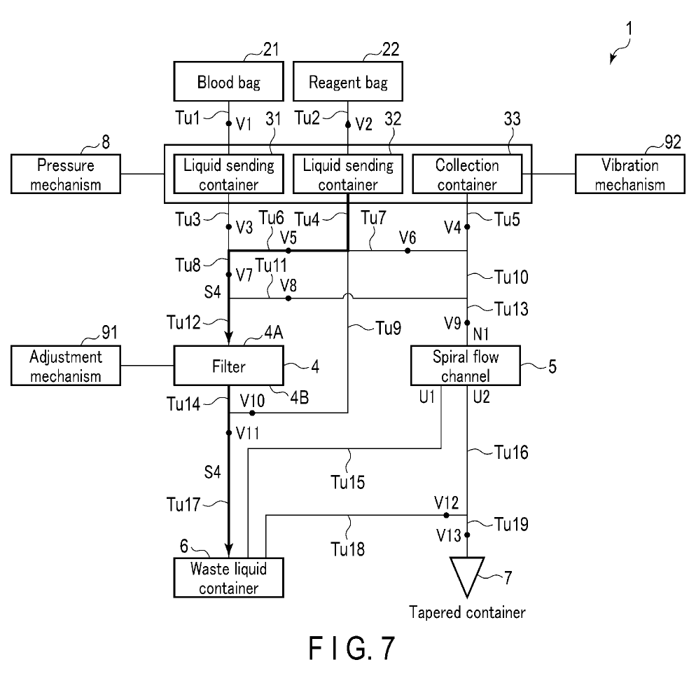

(Step S4) Subsequently, the particle separation apparatus 1 performs PBS rinsing (see FIG. 7). More specifically, the particle separation apparatus 1 sets the valves V5, V7, and V11 to the “open state”, and sets the valves V1 to V4, V6, V8 to V10, and V12 and V13 to the “closed state”. The particle separation apparatus 1 controls the pressure mechanism 8 to pressurize the liquid sending container 32. Accordingly, the PBS stored in the liquid sending container 32 is sent to the trapping surface 4A of the filter 4 through the tubes Tu4, Tu6, Tu8, and Tu12. The PBS that has been sent washes away the red blood cells, the platelets, and the like remaining on the trapping surface 4A. The PBS that has passed through the trapping surface 4A is sent from the opposite surface 4B of the filter 4, and is sent to the waste liquid container 6 through the tubes Tu14 and Tu17.

The particle separation apparatus 1 may rinse the filter 4 at a first flow rate for a predetermined time from the start of sending of the PBS, and subsequently rinse the filter 4 at a second flow rate lower than the first flow rate. Thus, the particle separation apparatus 1 can effectively wash away the red blood cells, the platelets, and the like remaining in the filter 4.

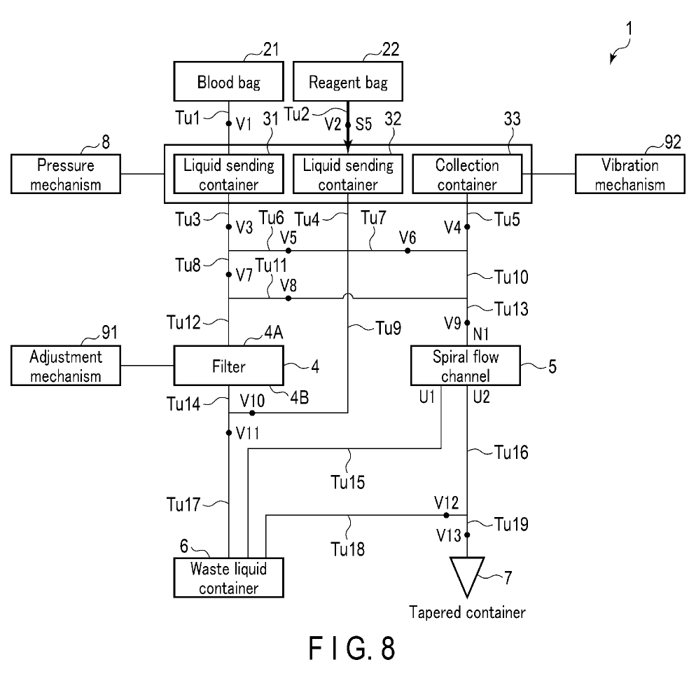

(Step S5) Subsequently, the particle separation apparatus 1 performs a secondary PBS suction (see FIG. 8). More specifically, the particle separation apparatus 1 sets the valve V2 to the “open state” and the valves V1 and V3 to V13 to the “closed state”. The particle separation apparatus 1 controls the pressure mechanism 8 to depressurize the liquid sending container 32. As a result, the PBS stored in the reagent bag 22 is sent to the liquid sending container 32 through the tube Tu2. Step S5 is similar to step S3.

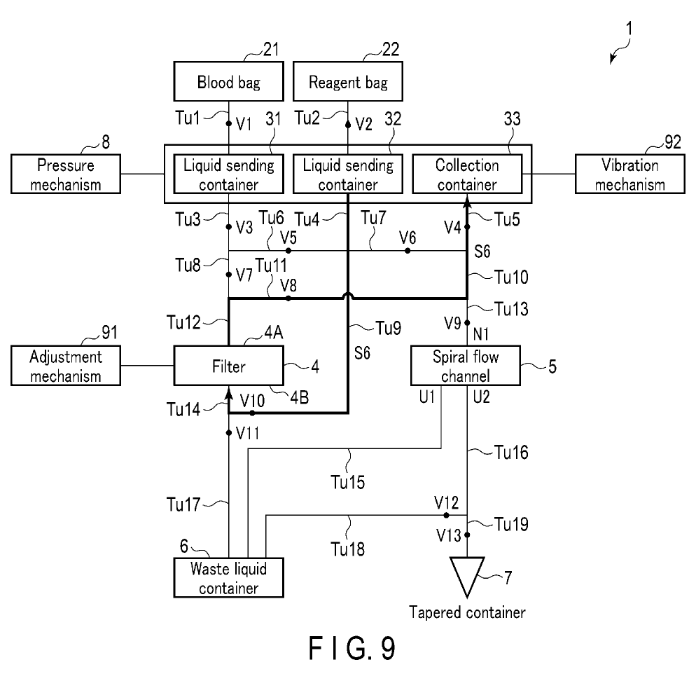

(Step S6) Subsequently, the particle separation apparatus 1 performs the collection of the cells (see FIG. 9). More specifically, the particle separation apparatus 1 sets the valves V4, V8, and V10 to the “open state”, and sets the valves V1 to V3, V5 to V7, V9, and V11 to V13 to the “closed state”. The particle separation apparatus 1 controls the pressure mechanism 8 to pressurize the liquid sending container 32. As a result, the PBS stored in the liquid sending container 32 is sent to the opposite surface 4B of the filter 4 through the tubes Tu4, Tu9, and Tu14. The sent PBS collects the suspension liquid containing the white blood cells caught on the trapping surface 4A from the direction opposite to the trapping surface 4A. The collected suspension liquid is sent to the collection container 33 through the tubes Tu12, Tu11, Tu10, and Tu5.

Prior to the collection of the suspension liquid, a vent (not shown) provided in the collection container 33 may be opened. The collection container 33 is depressurized by opening the vent. Thus, the suspension liquid collected from the filter 4 is efficiently collected into the collection container 33 against gravity.

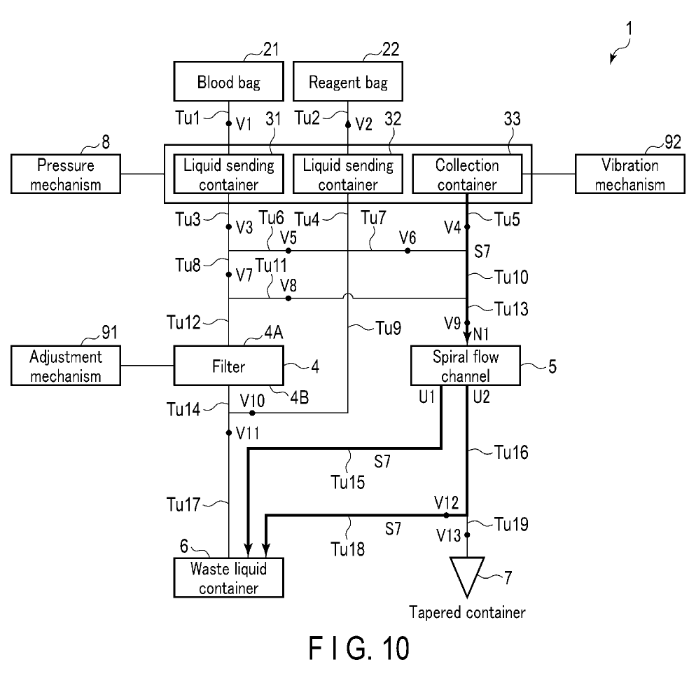

(Step S7) Subsequently, the particle separation apparatus 1 performs a primary classification (see FIG. 10). More specifically, the particle separation apparatus 1 sets the valves V4, V9, and V12 to the “open state”, and sets the valves V1 to V3, V5 to V8, V10 and V11, and V13 to the “closed state”. The particle separation apparatus 1 controls the pressure mechanism 8 to pressurize the collection container 33. Thus, the suspension liquid collected in the collection container 33 is sent to the inlet N1 of the spiral flow channel 5 through the tubes Tu5, Tu10, and Tu13. The suspension liquid mainly containing the red blood cells, the platelets, and the like is sent out from the outlet U1 of the spiral flow channel 5. The sent suspension liquid is sent to the waste liquid container 6 through the tube Tu15. The suspension liquid mainly containing the white blood cells is sent out from the outlet U2 of the spiral flow channel 5. The sent suspension liquid is sent to the waste liquid container 6 through the tubes Tu16 and Tu18.

The particle separation apparatus 1 may perform the primary classification until a flow amount of the suspension liquid sent to the inlet N1 of the spiral flow channel 5 reaches a predetermined value. After the flow rate has reached the predetermined value, the particle separation apparatus 1 performs a secondary classification.

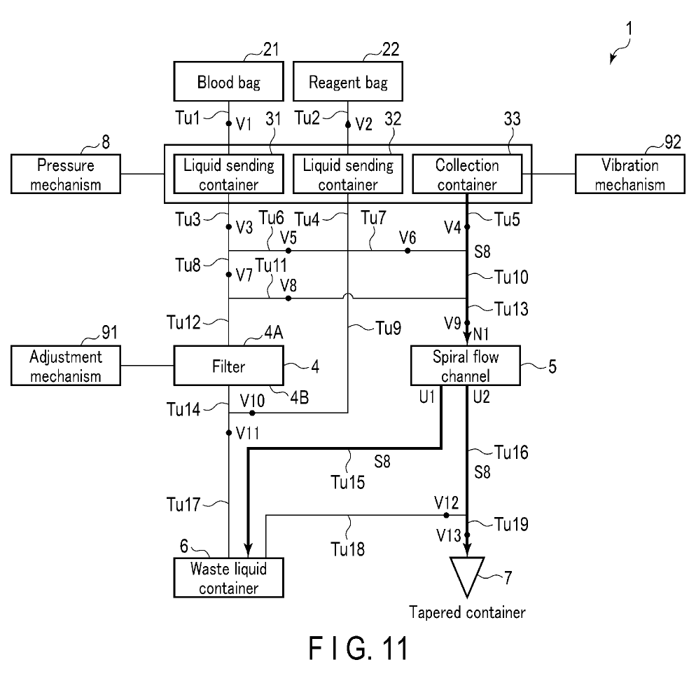

(Step S8) Finally, the particle separation apparatus 1 performs the secondary classification (see FIG. 11). More specifically, the particle separation apparatus 1 sets the valves V4, V9, and V13 to the “open state”, and sets the valves V1 to V3, V5 to V8, and V10 to V12 to the “closed state”. The particle separation apparatus 1 controls the pressure mechanism 8 to pressurize the collection container 33. Thus, the suspension liquid collected in the collection container 33 is sent to the inlet N1 of the spiral flow channel 5 through the tubes Tu5, Tu10, and Tu13. The suspension liquid mainly containing the red blood cells, the platelets, and the like is sent from the outlet U1 of the spiral flow channel 5. The sent suspension liquid is sent to the waste liquid container 6 through the tube Tu15. The suspension liquid mainly containing the white blood cells is sent from the outlet U2 of the spiral flow channel 5. The sent suspension liquid is sent to the tapered container 7 through the tubes Tu16 and Tu19.

According to the present embodiment described above, the particle separation apparatus 1 sends the blood to the filter 4, to roughly remove the red blood cells, the platelets, and the like in the blood. The particle separation apparatus 1 sends the PBS to the filter 4, thus collecting the suspension liquid mainly containing the white blood cells caught by the filter 4 in the collection container 33. The particle separation apparatus 1 sends the suspension liquid collected in the collection container 33 to the spiral flow channel 5, to further remove the red blood cells, the platelets, and the like in the suspension liquid and to separate the white blood cells. According to the implementation by the present inventors, in a case where the cell suspension liquid after separation was analyzed with a flow cytometer, it was observed that about 90% or more of the obtained cells were composed of the white blood cells.

The particle separation apparatus 1 does not perform, for example, a dilution process on the blood. The particle separation apparatus 1 can adjust the number of cells in the suspension liquid sent to the spiral flow channel 5 to the number of cells suitable for the spiral flow channel 5 in a short time by passing the blood through the filter 4. Thus, the particle separation apparatus 1 can efficiently separate the white blood cells from a large amount of blood (e.g., 10 mL). Furthermore, by causing the blood to pass through the filter 4, the particle separation apparatus 1 can adjust the number of cells without using a chemical substance that may damage the cells or may cause a concern, for example.

The suspension liquid stored in the blood bag 21 or the liquid sending container 31 may be referred to as a first suspension liquid. The suspension liquid sent to the spiral flow channel 5 may be referred to as a second suspension liquid. A ratio between the number of the non-target particles in the first suspension liquid and the number of the non-target particles in the second suspension liquid may be 1000 to 1. In this case, the spiral flow channel 5 can more effectively remove the non-target particles from the suspension liquid.

Note that a microchannel having any shape (e.g., linear, bellows-like, or curved) may be used instead of the spiral flow channel 5. In the above-described embodiment, the step of sending the suspension liquid containing the target particles and the non-target particles smaller than the target particles to the filter 4 is performed via the pressure mechanism 8, but this is not necessarily required. In the above-described embodiment, the step of sending the suspension liquid containing the target particles caught by the filter 4 to the microchannel is performed via the pressure mechanism 8, but this is not necessarily required.

According to at least one of the embodiments described above, the target particles can be efficiently separated.

While certain embodiments have been described, these embodiments have been presented by way of example only, and are not intended to limit the scope of the inventions. These embodiments can be implemented in various other forms, and various omissions, replacements, changes, and combinations of the embodiments can be made without departing from the gist of the invention. The embodiments and their modifications are included in the scope and gist of the invention, and are also included in the invention described in the scope of the claims and their equivalents.

With respect to the above embodiments, the following additional notes are disclosed as aspects and selective features of the inventions.

### Additional Note 1

A particle separation apparatus comprising: a reservoir configured to store a suspension liquid containing target particles and non-target particles smaller than the target particles;

a filter configured to catch the target particles from the suspension liquid and allow the non-target particles to pass through; and

a microchannel configured to separate the target particles from the suspension liquid,

wherein the suspension liquid stored in the reservoir is sent to the filter, and the suspension liquid containing the target particles caught by the filter is sent to the microchannel.

### Additional Note 2

The filter may have a trapping surface configured to catch the target particles.

The particle separation apparatus may collect the suspension liquid containing the target particles caught at the trapping surface by sending a liquid to the trapping surface from a direction opposite to the trapping surface.

### Additional Note 3

The particle separation apparatus may include a first tube configured to connect the reservoir and the trapping surface.

The particle separation apparatus may include a second tube configured to connect the trapping surface and the microchannel.

The particle separation apparatus may send the suspension liquid to the trapping surface through the first tube.

The particle separation apparatus may send the suspension liquid to the microchannel through the second tube.

### Additional Note 4

The particle separation apparatus may send the suspension liquid to the filter at a first flow rate for a predetermined time from a start of sending the suspension liquid, and subsequently send the suspension liquid to the filter at a second flow rate lower than the first flow rate.

### Additional Note 5

The particle separation apparatus may rinse the filter with a liquid.

The particle separation apparatus may send the suspension liquid containing the target particles caught by the rinsed filter to the microchannel.

### Additional Note 6

The particle separation apparatus may rinse the filter at a first flow rate for a predetermined time from a start of sending the liquid, and subsequently rinse the filter at a second flow rate lower than the first flow rate.

### Additional Note 7

The particle separation apparatus may include a collection container configured to store the suspension liquid containing the target particles caught by the filter.

The particle separation apparatus may send the suspension liquid stored in the collection container to the microchannel.

### Additional Note 8

The particle separation apparatus may include a vibration mechanism configured to vibrate the collection container.

The vibration mechanism may vibrate the suspension liquid stored in the collection container.

The particle separation apparatus may send the vibrated suspension liquid to the microchannel.

### Additional Note 9

The waste liquid container may store the suspension liquid containing the non-target particles that have passed through the filter.

The waste liquid container may store the suspension liquid containing the non-target particles separated by the microchannel.

### Additional Note 10

The particle separation apparatus may include an adjustment mechanism configured to adjust a posture of the filter for installation.

The adjustment mechanism may adjust the posture to be horizontal.

The particle separation apparatus may send the suspension liquid vertically downward to the filter adjusted to be horizontal.

### Additional Note 11

The target particles may each have a particle size of 7 μm or more and 30 μm or less.

The non-target particles may each have a particle size of less than 7 μm.

### Additional Note 12

The target particles may be white blood cells.

### Additional Note 13

The suspension liquid stored in the reservoir may be a first suspension liquid.

The suspension sent to the microchannel may be a second suspension liquid.

A ratio between the number of the non-target particles in the first suspension liquid and the number of the non-target particles in the second suspension liquid may be 1000 to 1.

### Additional Note 14

The microchannel may be a spiral microchannel.

### Additional Note 15

A particle separation method comprising: a step of sending a suspension liquid containing target particles and non-target particles smaller than the target particles to a filter;

a step of catching the target particles from the suspension liquid by the filter and allowing the non-target particles to pass through the filter;

a step for sending the suspension liquid containing the target particles caught by the filter to the microchannel; and

a step in which the microchannel separates the target particles from the suspension liquid.

### Additional Note 16

The filter may have a trapping surface configured to catch the target particles.

The particle separation method may include a step of collecting the suspension liquid containing the target particles caught at the trapping surface by sending a liquid to the trapping surface from a direction opposite to the trapping surface.

### Additional Note 17

The particle separation method may include a step of sending the suspension liquid to the filter at a first flow rate for a predetermined time from a start of sending the suspension liquid, and subsequently a step of sending the suspension liquid to the filter at a second flow rate lower than the first flow rate.

### Additional Note 18

The particle separation method may include a step of rinsing the filter with a liquid.

The particle separation method may include a step of sending the suspension liquid containing the target particles caught by the rinsed filter to the microchannel.

### Additional Note 19

The particle separation method may include a step of rinsing the filter at a first flow rate for a predetermined time from a start of sending the liquid, and subsequently rinsing the filter at a second flow rate lower than the first flow rate.

### Additional Note 20

The particle separation method may include a step of adjusting an installation posture of the filter to be horizontal.

The particle separation method may include a step of sending the suspension liquid vertically downward to the filter adjusted to be horizontal.

What is claimed is:

1. A particle separation apparatus comprising:
a container configured to store a suspension liquid containing target particles and non-target particles smaller than the target particles;
a filter configured to catch the target particles from the suspension liquid and allow the non-target particles to pass through; and
a microchannel configured to separate the target particles from the suspension liquid,
wherein the suspension liquid stored in the container is sent to the filter, and the suspension liquid containing the target particles caught by the filter is sent to the microchannel.

2. The particle separation apparatus according to claim 1, wherein
the filter includes a trapping surface configured to catch the target particles, and
the suspension liquid containing the target particles caught at the trapping surface is collected by sending a liquid to the trapping surface from a direction opposite to the trapping surface.

3. The particle separation apparatus according to claim 2, further comprising:
a first tube configured to connect the container and the trapping surface; and
a second tube configured to connect the trapping surface and the microchannel,
wherein the suspension liquid is sent to the trapping surface through the first tube, and the suspension liquid is sent to the microchannel through the second tube.

4. The particle separation apparatus according to claim 1, wherein
the suspension liquid is sent to the filter at a first flow rate for a predetermined time from a start of sending the suspension liquid, and the suspension liquid is subsequently sent to the filter at a second flow rate lower than the first flow rate.

5. The particle separation apparatus according to claim 1, wherein
the filter is rinsed with a liquid, and the suspension liquid containing the target particles caught by the rinsed filter is sent to the microchannel.

6. The particle separation apparatus according to claim 5, wherein
the filter is rinsed at a first flow rate for a predetermined time from a start of sending the liquid, and the filter is subsequently rinsed at a second flow rate lower than the first flow rate.

7. The particle separation apparatus according to claim 1, further comprising:
a collection container configured to store the suspension liquid containing the target particles caught by the filter,
wherein the suspension liquid stored in the collection container is sent to the microchannel.

8. The particle separation apparatus according to claim 7, further comprising:
a base and a vibration device configured to vibrate the collection container,
wherein the base and the vibration device vibrate the suspension liquid stored in the collection container, and
send the vibrated suspension liquid to the microchannel.

9. The particle separation apparatus according to claim 1, further comprising:
a waste liquid container configured to store the suspension liquid containing the non-target particles that have passed through the filter and to store the suspension liquid containing the non-target particles separated by the microchannel.

10. The particle separation apparatus according to claim 1, further comprising:
a stage and a drive mechanism configured to adjust a posture of the filter for installation,
wherein the stage and the drive mechanism adjust the posture to be horizontal, and
the suspension liquid is sent vertically downward to the filter adjusted to be horizontal.

11. The particle separation apparatus according to claim 1, wherein
the target particles each have a particle size of 7 μm or more and 30 μm or less, and
the non-target particles each have a particle size of less than 7 μm.

12. The particle separation apparatus according to claim 1,
wherein the target particles are white blood cells.

13. The particle separation apparatus according to claim 1, wherein
the suspension liquid stored in the container is a first suspension liquid,
the suspension liquid sent to the microchannel is a second suspension liquid, and
a ratio between the number of the non-target particles in the first suspension liquid and the number of the non-target particles in the second suspension liquid is 1000 to 1.

14. The particle separation apparatus according to claim 1,
wherein the microchannel is a spiral microchannel.

15. A particle separation method, comprising:
sending a suspension liquid containing target particles and non-target particles smaller than the target particles to a filter;
catching the target particles from the suspension liquid by the filter and allowing the non-target particles to pass through the filter;
sending the suspension liquid containing the target particles caught by the filter to a microchannel; and
separating the target particles from the suspension liquid by the microchannel.

16. The particle separation method according to claim 15, wherein
the filter includes a trapping surface configured to catch the target particles, and
the method further comprises collecting the suspension liquid containing the target particles caught at the trapping surface by sending a liquid to the trapping surface from a direction opposite to the trapping surface.

17. The particle separation method according to claim 15, further comprising:
sending the suspension liquid to the filter at a first flow rate for a predetermined time from a start of sending the suspension liquid, and subsequently sending the suspension liquid to the filter at a second flow rate lower than the first flow rate.

18. The particle separation method according to claim 15, further comprising:
rinsing the filter with a liquid; and
sending the suspension liquid containing the target particles caught by the rinsed filter to the microchannel.

19. The particle separation method according to claim 18, further comprising:
rinsing the filter at a first flow rate for a predetermined time from a start of sending the liquid, and subsequently rinsing the filter at a second flow rate lower than the first flow rate.

20. The particle separation method according to claim 15, further comprising:
adjusting an installation posture of the filter to be horizontal; and
sending the suspension liquid vertically downward to the filter adjusted to be horizontal.
